(19) United States
(12) Patent Application Publication
(10) Pub. No.: US 2023/0408761 A1
(43) Pub. Date: Dec. 21, 2023

(54) ULTRAVIOLET LIGHT IRRADIATION SYSTEM AND METHOD

(71) Applicant: NIPPON TELEGRAPH AND TELEPHONE CORPORATION, Tokyo (JP)

(72) Inventors: Tomohiro TANIGUCHI, Musashino-shi, Tokyo (JP); Ayako IWAKI, Musashino-shi, Tokyo (JP); Kazuhide NAKAJIMA, Musashino-shi, Tokyo (JP); Nobutomo HANZAWA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP)

(73) Assignee: NIPPON TELEGRAPH AND TELEPHONE CORPORATION, Tokyo (JP)

(21) Appl. No.: 18/032,767

(22) PCT Filed: Oct. 22, 2020
(86) PCT No.: PCT/JP2020/039661

§ 371 Date: Apr. 19, 2023

(51) Int. Cl. G02B 6/04; A61L 2/10

(52) U.S. Cl. CPC G02B 6/04; A61L 2202/11; A61L 2/10

(57) ABSTRACT

An object of the present disclosure is to alleviate deterioration of transmission characteristics of a fiber due to transmission of ultraviolet light, and to eliminate complication of operation due to frequent replacement of an optical fiber in which deterioration has occurred.

The present disclosure is an ultraviolet light irradiation system including: an optical transmission unit that propagates ultraviolet light using a plurality of optical transmission lines; an ultraviolet light source unit that inputs ultraviolet light to each of the optical transmission lines with arbitrary power; and an irradiation unit that irradiates a target location with the ultraviolet light propagated through the plurality of optical transmission lines.

### TECHNICAL FIELD

The present disclosure relates to sterilization using ultraviolet light.

### BACKGROUND ART

Conventional sterilization using ultraviolet light includes an autonomous mobile robot that emits ultraviolet light, a stationary air purifier that is installed at a predetermined indoor place and sterilizes indoor air while circulating the air, and a portable sterilizer equipped with an ultraviolet light source. However, conventional sterilization using ultraviolet light has problems that it is large-scale and expensive, that direct irradiation to a necessary place cannot be performed, and that high skill is required in use.

On the other hand, a system using a thin and bendable optical fiber is considered (e.g., refer to Non Patent Literature 1). However, in a case where an optical fiber is used for transmission of ultraviolet light, there is a problem that transmission characteristics of the optical fiber deteriorate. Specifically, by transmitting high-energy light in the ultraviolet region, defects occur in core glass, and the transmission loss characteristics deteriorate over time.

### CITATION LIST

### Non Patent Literature

Non Patent Literature 1: Baba et al., “Development of UV-Dedicated Heat-Resistant Optical Fibers (2)”, MITSUBISHI CABLE INDUSTRIES, LTD. Current News, No. 100, pp. 84-88, April 2003

### SUMMARY OF INVENTION

### Technical Problem

An object of the present disclosure is to alleviate deterioration of transmission characteristics of a fiber due to transmission of ultraviolet light, and to eliminate complication of operation due to frequent replacement of an optical fiber in which deterioration has occurred.

### Solution to Problem

An ultraviolet light irradiation system according to the present disclosure includes:
an optical transmission unit that propagates ultraviolet light using a plurality of optical transmission lines;
an ultraviolet light source unit that inputs ultraviolet light to each of the optical transmission lines with arbitrary power; and
an irradiation unit that irradiates a target location with the ultraviolet light propagated through the plurality of optical transmission lines.

An ultraviolet light irradiation method according to the present disclosure includes:
propagating ultraviolet light to a single irradiation unit using a plurality of optical transmission lines
when irradiating a target location with the ultraviolet light outputted from an ultraviolet light source unit by an irradiation unit.

### Advantageous Effects of Invention

It is possible with the present disclosure to alleviate deterioration of transmission characteristics of a fiber due to transmission of ultraviolet light, and eliminate complication of operation due to frequent replacement of an optical fiber in which deterioration has occurred.

### BRIEF DESCRIPTION OF DRAWINGS

FIG. 1 illustrates an example of a system configuration of the present disclosure.

FIG. 2 illustrates a configuration example of an optical transmission line of the present disclosure.

FIG. 3 illustrates a configuration example of an optical transmission line of the present disclosure.

FIG. 4 illustrates a configuration example of an optical transmission line of the present disclosure.

FIG. 5A illustrates an example of a configuration pattern of a single-core optical fiber.

FIG. 5B illustrates an example of a configuration pattern of a single-core optical fiber.

FIG. 5C illustrates an example of a configuration pattern of a single-core optical fiber.

FIG. 5D illustrates an example of a configuration pattern of a single-core optical fiber.

FIG. 5E illustrates an example of a configuration pattern of a single-core optical fiber.

FIG. 6A illustrates an example of a configuration pattern of a multi-core optical fiber.

FIG. 6B illustrates an example of a configuration pattern of a multi-core optical fiber.

FIG. 6C illustrates an example of a configuration pattern of a multi-core optical fiber.

FIG. 6D illustrates an example of a configuration pattern of a multi-core optical fiber.

FIG. 6E illustrates an example of a configuration pattern of a multi-core optical fiber.

FIG. 7A illustrates an example of a configuration of an ultraviolet light source unit.

FIG. 7B illustrates an example of a configuration of an ultraviolet light source unit.

FIG. 8 illustrates an example of a system configuration of the present disclosure.

FIG. 9 illustrates a configuration example of an optical distribution unit.

FIG. 10 illustrates an example of a system configuration of the present disclosure.

FIG. 11 illustrates an example of a system configuration of the present disclosure.

### DESCRIPTION OF EMBODIMENTS

The following description will explain embodiments of the present disclosure in detail with reference to the drawings. Note that the present disclosure is not limited to the embodiments described below. These examples are merely illustrations, and the present disclosure can be carried out in forms with various modifications and improvements based on the knowledge of those skilled in the art. Note that components having the same reference numerals in the present specification and the drawings indicate the same components.

### First Embodiment

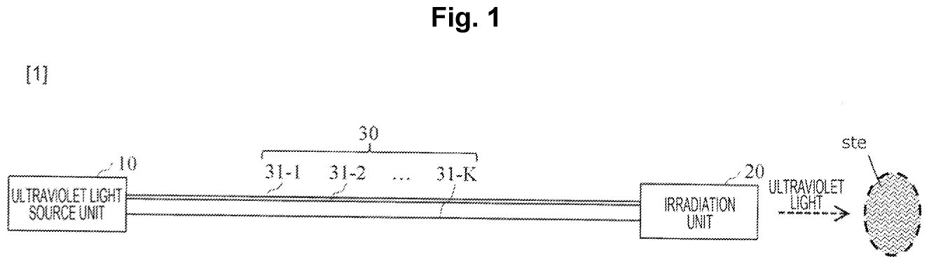

FIG. 1 illustrates an example of a system configuration of the present embodiment. An ultraviolet light irradiation system of the present embodiment includes:
an ultraviolet light source unit 10 that generates ultraviolet light;
an irradiation unit 20 that irradiates a target location ste with the ultraviolet light; and
an optical transmission unit 30 that propagates ultraviolet light from the ultraviolet light source unit 10 to the irradiation unit 20.

The optical transmission unit 30 has K cores 31-1, 31-2, . . . , and 31-K that function as optical transmission lines. The ultraviolet light source unit 10 inputs ultraviolet light to each of the cores 31-1, 31-2, . . . , and 31K included in the optical transmission unit 30 at an arbitrary timing and with an arbitrary power. The number K of the cores 31 is an arbitrary number of 2 or more. In the present disclosure, the cores 31-1, 31-2, . . . , and 31-K are referred to as cores 31 in a case where it is unnecessary to distinguish therebetween.

The irradiation unit 20 irradiates the target location ste to be sterilized with the ultraviolet light transmitted by each core 31. The irradiation unit 20 has an arbitrary configuration capable of irradiating the target location ste, and includes, for example, an optical system such as a lens designed to transmit a wavelength in an ultraviolet region.

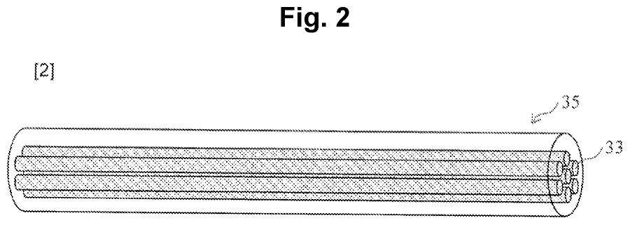

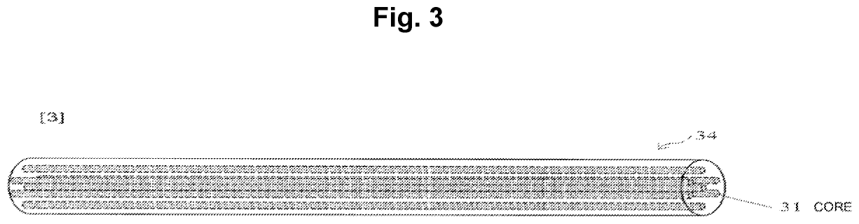

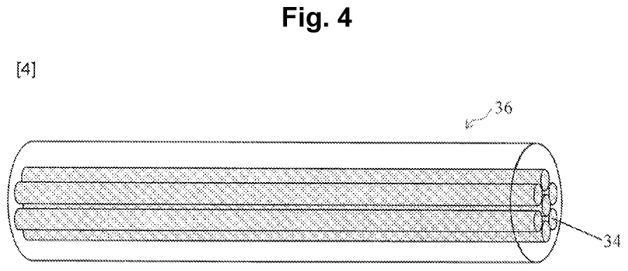

FIGS. 2 to 4 each illustrate a configuration example of the optical transmission unit 30. As the optical transmission unit 30, an arbitrary form having a plurality of cores 31 can be used. For example, an optical cable 35 having a plurality of single-core optical fibers 33 as illustrated in FIG. 2, a multi-core optical fiber 34 having a plurality of cores 31 as illustrated in FIG. 3, or an optical cable 36 having a plurality of multi-core optical fibers 34 as illustrated in FIG. 4 can be exemplified. The optical cable 35 functions as a first optical cable, and the optical cable 36 functions as a second optical cable. Note that the optical fibers in the optical cables 35 and 36 may have a tape shape. Moreover, a single-core optical fiber and a multi-core optical fiber may be provided in one cable.

The optical transmission unit 30 transmits ultraviolet light to each irradiation unit 20 using the plurality of cores 31. Since the optical transmission unit 30 of the present disclosure is thin and bendable, it can be laid in a small place where a conventional robot/device cannot enter.

The single-core optical fiber 33 is an optical fiber having one core 31 which is a waveguide region. The multi-core optical fiber is an optical fiber having at least two or more waveguide regions, in which the waveguide regions are selectively used (multi-core optical fiber or coupled type multi-core optical fiber). As described above, in the present disclosure, a plurality of optical transmission lines is configured using one or more waveguide regions provided in an optical fiber.

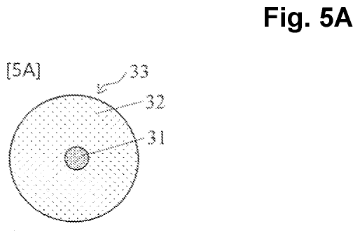

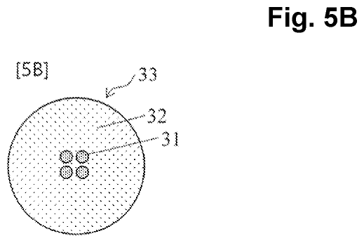

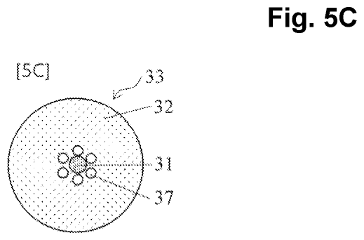

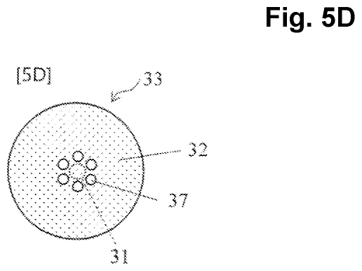

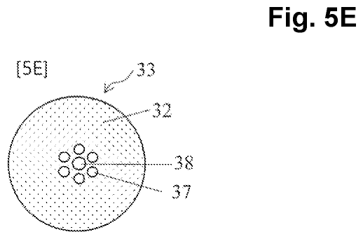

A configuration example of the single-core optical fiber 33 is described in FIGS. 5A to 5E.

The single-core optical fiber 33 is, for example, a solid core type optical fiber in which a waveguide region is constituted with a single core 31 having a refractive index higher than that of a clad 32 as illustrated in FIG. 5A. “Solid” means “not hollow”. Furthermore, the solid core can also be made by forming an annular low refractive index region in the clad.

The single-core optical fiber 33 is, for example, a coupled-core type optical fiber in which a waveguide region is configured with at least two or more cores 31 having inter-core coupling and light is guided by optical wave coupling between the plurality of cores 31 as illustrated in FIG. 5B.

The single-core optical fiber 33 is, for example, a hole-assist type optical fiber in which a waveguide region is configured with one independent core 31 and a plurality of holes 37 arranged at equal intervals on the outer periphery of the one core 31 as illustrated in FIG. 5C. The medium of the hole is air, and the refractive index of air is sufficiently smaller than that of quartz-based glass. Therefore, the hole-assist type optical fiber has a function of returning light leaked from the core by bending or the like to the core again, and is characterized by having a small bending loss.

The single-core optical fiber 33 is, for example, a hole-structure optical fiber in which a waveguide region is configured with a plurality of holes 37 provided in the clad 32 and the clad 32 surrounded by the plurality of holes 37 functions as the core 31 as illustrated in FIG. 5D. This structure is called a photonic crystal fiber. In this structure, it is possible to adopt a structure including no high refractive index core having a changed refractive index, and it is possible to confine light by using a region surrounded by holes as an effective core region. Compared with an optical fiber having a solid core, the photonic crystal fiber can reduce the influence of absorption and scattering loss due to additives in the core, and can realize optical characteristics that cannot be realized by a solid optical fiber, such as reduction of bending loss and control of a nonlinear effect.

The single-core optical fiber 33 is, for example, a hollow core type hole-structure optical fiber in which a waveguide region guides light to a cavity 38 surrounded by the holes 37 provided in the clad 32 as illustrated in FIG. 5E. In this optical fiber, a core region is formed of air. Light can be confined in the core region by adopting a photonic band gap structure configured with a plurality of holes or an anti-resonance structure configured with a thin glass wire in the clad region. This optical fiber has a small nonlinear effect, and can supply a high-power or high-energy laser.

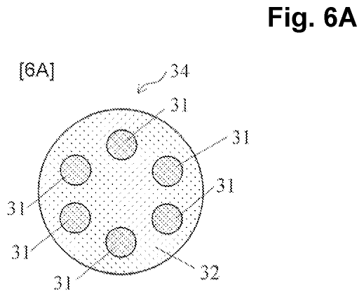

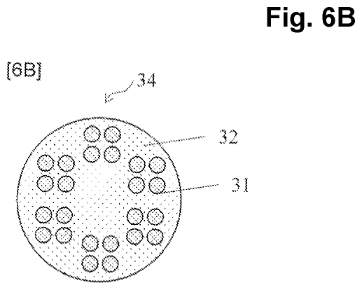

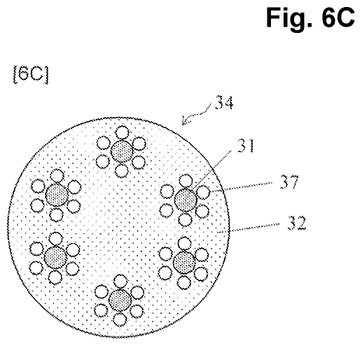

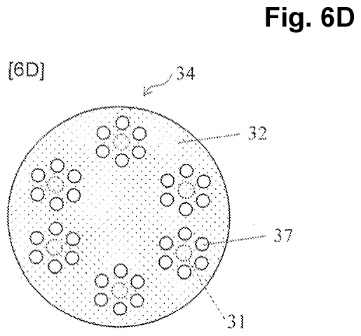

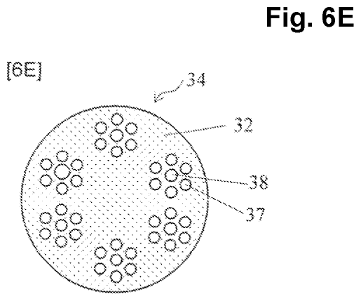

A configuration example of a multi-core optical fiber 34 having six waveguide regions is described in FIGS. 6A to 6E. The multi-core optical fiber 34 has a structure in which a plurality of at least one of the waveguide regions illustrated in FIGS. 5A to 5E is arranged in the same optical fiber cross section.

The multi-core optical fiber is, for example, an optical fiber in which each waveguide region is configured with one independent core 31 as illustrated in FIG. 6A. This optical fiber guides light in a state where the influence of optical wave coupling can be ignored by sufficiently reducing the optical wave coupling between solid cores 52.

The multi-core optical fiber is, for example, a coupled-core type multi-core optical fiber in which each waveguide region is configured with at least two or more cores 31 having inter-core coupling as illustrated in FIG. 6B.

The multi-core optical fiber is, for example, a hole-assist type multi-core optical fiber in which each waveguide region is configured with one independent core 31 and a plurality of holes 37 arranged at equal intervals on the outer periphery of the one core 31 as illustrated in FIG. 6C.

The multi-core optical fiber is, for example, a hole-structure multi-core optical fiber in which each waveguide region is configured with a plurality of holes 37 provided in the clad 32, and the clad 32 surrounded by the plurality of holes 37 functions as the core 31 as illustrated in FIG. 6D.

The multi-core optical fiber is, for example, a hollow core type hole-structure multi-core optical fiber in which each waveguide region is configured with a cavity 38 surrounded by holes 37 provided in the clad 32 as illustrated in FIG. 6E.

Note that the number of cores of the multi-core optical fiber 34 may be an arbitrary number of 2 or more.

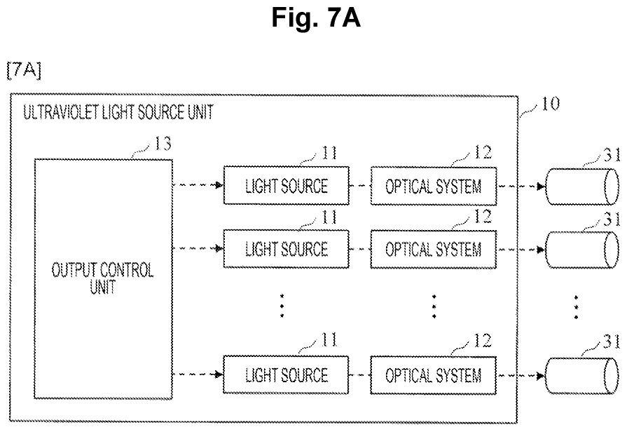

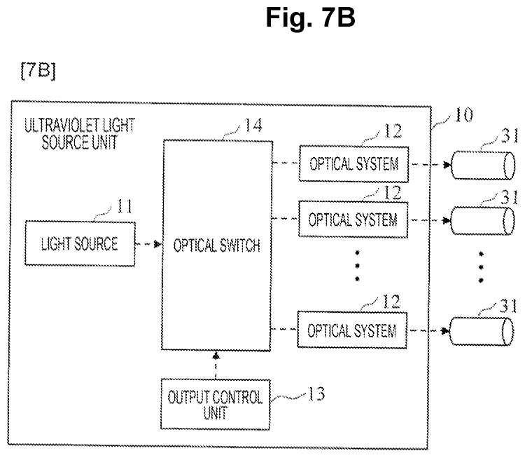

The configuration of the ultraviolet light source unit 10 will be described with reference to FIGS. 7A and 7B. The ultraviolet light source unit 10 outputs light including an ultraviolet region effective for inactivation and decomposition of bacteria and viruses. The wavelength outputted from the ultraviolet light source unit 10 is not limited to the ultraviolet region, and may include a wavelength region visually recognizable by a person, such as white light. The ultraviolet light source unit 10 has parameters for an output, a wavelength, and a waveform (such as a pulse), and outputs ultraviolet light having an output, a wavelength, and a waveform according to the parameters.

FIG. 7A illustrates an example in which the ultraviolet light source unit 10 is configured with a plurality of light sources 11. The ultraviolet light source unit 10 includes the plurality of light sources 11, a plurality of optical systems 12, and an output control unit 13. Each optical system 12 inputs output light of each light source 11 to the core 31 by using an optical system such as a lens. Each core 31 is one single-core optical fiber 33 in an optical cable 35, or one core 31 in a multi-core optical fiber 34.

FIG. 7B illustrates an example in which the ultraviolet light source unit 10 is configured with a single light source 11. The ultraviolet light source unit 10 includes the single light source 11, an optical switch 14 that controls the output of the single light source 11, a plurality of optical systems 12, and an output control unit 13. The optical switch 14 has a plurality of output ports, and outputs ultraviolet light inputted from the light source 11 to an output port according to the control from the output control unit 13.

The light source 11 is an arbitrary unit capable of outputting light in an ultraviolet region, and a semiconductor light source such as an LD or an LED, a light source using nonlinear optics, or a lamp light source can be used. The output control unit 13 performs on/off control and power control of ultraviolet light transmission to each core 31 by the following method.
Control is performed such that the ultraviolet light is outputted to each core 31 in a constant cycle and round robin.
The on/off and the power of the ultraviolet light output to each core 31 are controlled according to the situation of the target location ste to be sterilized/inactivated.

Note that the input power to each core 31 may be the same or different. Moreover, the optical system 12 may include an isolator that prevents return light from the core 31 from returning to the light source 11. Moreover, the optical fiber used for the optical transmission unit 30 of the present embodiment may be a large-diameter multimode fiber capable of transmitting high energy.

### Second Embodiment

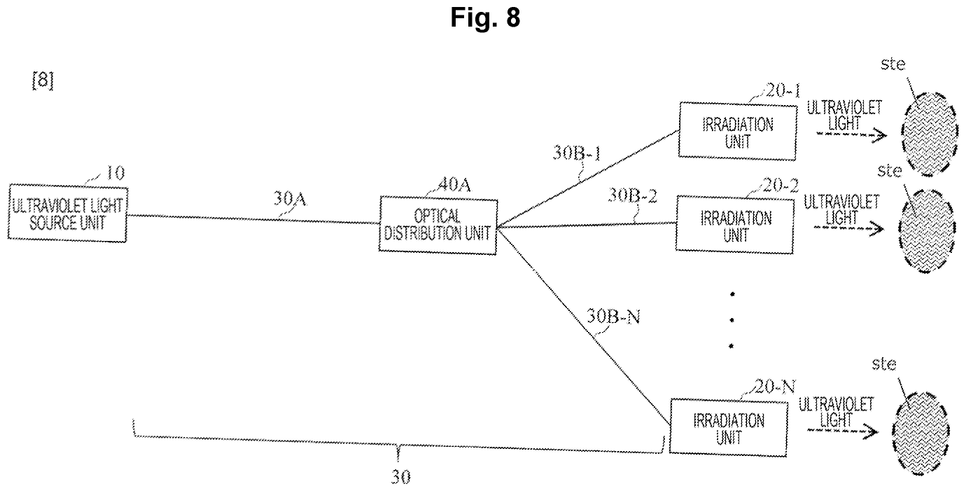

FIG. 8 illustrates an example of a system configuration of the present embodiment. In the ultraviolet light irradiation system of the present embodiment, an optical transmission unit 30 includes an optical distribution unit 40A that functions as a first optical distribution unit.

An ultraviolet light source unit 10 and the optical distribution unit 40A are connected by an optical transmission unit 30A, and the optical distribution unit 40A is connected with N irradiation units 20-1, . . . , and 20N by N optical transmission units 30B-1, . . . , and 30B-N. In the present disclosure, the irradiation units 20-1, . . . , and 20-N will be described as irradiation units 20 in a case where it is unnecessary to distinguish therebetween, and the optical transmission units 30B-1, . . . , and 30B-N will be described as optical transmission units 30B in a case where it is unnecessary to distinguish therebetween.

The optical distribution unit 40A distributes the ultraviolet light propagated by each core 31 included in the optical transmission unit 30A into N parts for each core 31. Therefore, the optical transmission unit 30B includes a plurality of cores 31 similarly to the optical transmission unit 30A. Similarly to the optical transmission unit 30 described in the first embodiment, the optical transmission units 30A and 30B can be each configured with an optical cable 35 in which a plurality of single-core optical fibers 33 is bundled, a multi-core optical fiber 34 having a plurality of cores 31, or an optical cable 36 in which multi-core optical fibers 34 are bundled.

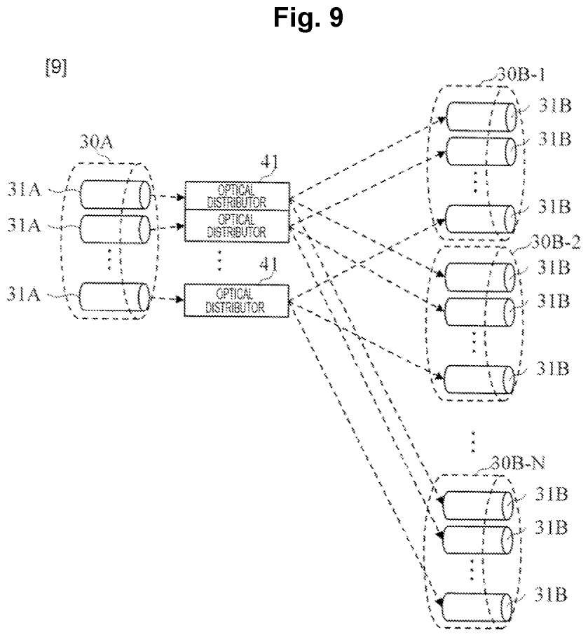

FIG. 9 illustrates a configuration example of the optical distribution unit 40A. The optical distribution unit 40A includes an optical distributor 41 for each core 31. Each optical distributor 41 branches the light from each core 31A included in the optical transmission unit 30A into N parts, and outputs the light to cores 31B included in each of the optical transmission units 30B-1 to 30B-N. As the optical distributor 41, an arbitrary device capable of branching ultraviolet light, such as an optical splitter, can be used.

The optical transmission units 30B-1, . . . , and 30B-N transmit ultraviolet light to the irradiation units 20-1, . . . , and 20N, respectively. The optical transmission units 30B-1, . . . , and 30B-N can be laid even in a small place where a conventional robot/device cannot enter.

### Third Embodiment

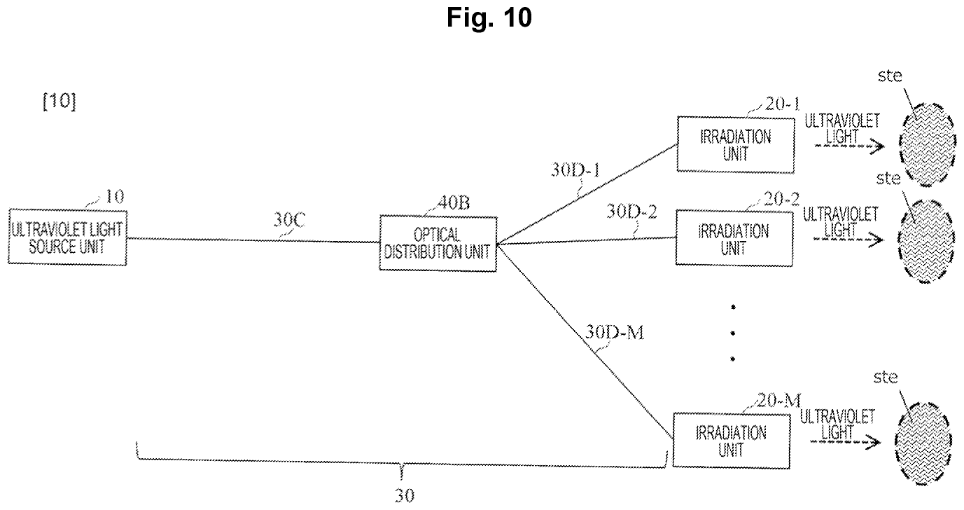

FIG. 10 illustrates an example of a system configuration of the present embodiment. In the ultraviolet light irradiation system of the present embodiment, an optical transmission unit 30 includes an optical distribution unit 40B that functions as a second optical distribution unit.

An ultraviolet light source unit 10 and the optical distribution unit 40B are connected by an optical transmission unit 30C, and the optical distribution unit 40B is connected with M irradiation units 20-1, . . . , and 20-M by M optical transmission units 30D-1, . . . , and 30D-M. In the present disclosure, the irradiation units 20-1, . . . , and 20-M will be referred to as irradiation units 20 in a case where it is unnecessary to distinguish therebetween, and the optical transmission units 30D-1, . . . , and 30D-M will be referred to as optical transmission units 30B in a case where it is unnecessary to distinguish therebetween.

In the present embodiment, the optical transmission unit 30C is an optical cable 36 in which a plurality of multi-core optical fibers 34 are bundled, and the optical transmission units 30D-1, . . . , and 30D-M are multi-core optical fibers 34. The optical distribution unit 40B fans out the multi-core optical fibers 34 included in the optical transmission unit 30C. Thus, the optical distribution unit 40B distributes the ultraviolet light transmitted from the ultraviolet light source unit 10 to a plurality of multi-core optical fibers.

For example, in a case where the optical transmission unit 30C includes M multi-core optical fibers 34, the M multi-core optical fibers in the optical cable provided in the optical transmission unit 30C on the input side of the optical distribution unit 40B and the respective multi-core optical fibers 34 provided in the optical transmission unit 30D on the output side of the optical distribution unit 40 are connected by 1:1. Note that the fan-out may provide a configuration in which the outer sheath of the optical cable is removed and each multi-core optical fiber is taken out.

The optical transmission units 30D-1, . . . , and 30D-M transmit ultraviolet light to the irradiation units 20-1, . . . , and 20-M, respectively. The optical transmission units 30C and 30D can be laid even in a small place where a conventional robot/device cannot enter. Note that the optical transmission units 30D-1, . . . , and 30D-M may be an optical cable 35 in which a plurality of single-core optical fibers 33 is bundled.

### Fourth Embodiment

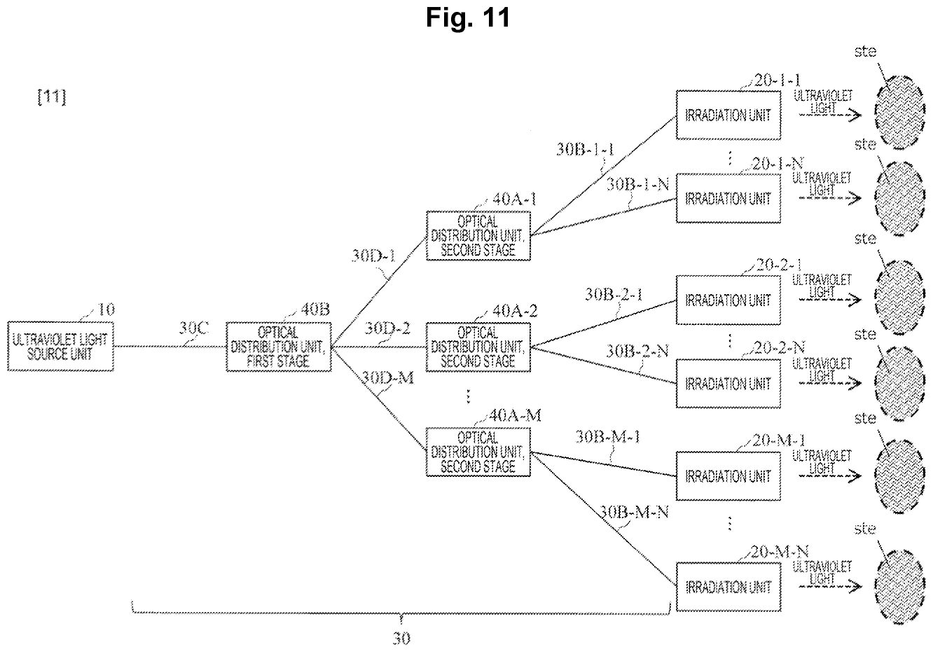

FIG. 11 illustrates an example of a system configuration of the present embodiment. The present embodiment has a configuration in which the second embodiment and the third embodiment are combined. Specifically, in an ultraviolet light irradiation system of the present embodiment, an optical transmission unit 30 includes optical distribution units 40A and 40B. An ultraviolet light source unit 10 and the optical distribution unit 40B are connected by an optical transmission unit 30C, the optical distribution unit 40B and the optical distribution units 40A are connected by optical transmission units 30D, and the optical distribution units 40A are connected with a plurality of irradiation units 20 by optical transmission units 30B.

The optical transmission unit 30C is an optical cable 36 in which a plurality of multi-core optical fibers 34 are bundled. Each optical transmission unit 30D is a multi-core optical fiber 34. Each optical transmission unit 30B is an optical cable 35 in which a plurality of single-core optical fibers 33 is bundled, or a multi-core optical fiber 34. Each optical transmission unit 30D may be an optical cable 35 in which a plurality of single-core optical fibers 33 is bundled.

Each optical transmission unit 30B transmits ultraviolet light to each irradiation unit 20. The optical transmission units 30C, 30D, and 30B can be laid in a small place where a conventional robot/device cannot enter.

As described above, the present disclosure has the following configuration.

In the system configuration, the ultraviolet light source unit 10 and the irradiation units 20 installed near target locations ste to be sterilized/inactivated are connected by an optical cable in which a plurality of optical fibers (single-core or multi-core) is bundled, or a multi-core optical fiber.

Furthermore, the ultraviolet light source unit 10 is configured to perform on/off control or power control of ultraviolet light to be transmitted to a plurality of optical fibers or cores.

As a result, a system of the present disclosure can alleviate the problem of deterioration in transmission characteristics of an optical fiber due to transmission of ultraviolet light, and can be efficiently operated.

### Effects of the Present Disclosure

In a sterilization/inactivation system using ultraviolet light, it is possible to realize a system capable of economically sterilizing/deactivating a desired location by utilizing an optical cable in which a plurality of optical fibers (single-core or multi-core) is bundled, or a multi-core optical fiber.

### REFERENCE SIGNS LIST

Ultraviolet light source unit
20, 20-1, 20-2, . . . , 20-M, 20-N Irradiation unit
30, 30A, 30B, 30B-1, 30B-2, 30B-N, 30C, 30D, 30D-1, 30D-2, 30D-M Optical transmission unit
31, 31-1, 31-2, . . . 31-K, 31A, 31B Core
32 Clad
33 Single-core optical fiber
34 Multi-core optical fiber
35, 36 Optical cable
37 Hole
40A, 40A-1, 40A-2, 40A-M, 40B Optical distribution unit
41 Optical distributor

1. An ultraviolet light irradiation system comprising:
an optical transmission unit that propagates ultraviolet light using a plurality of optical transmission lines;
an ultraviolet light source unit that inputs ultraviolet light to each of the optical transmission lines with arbitrary power; and
an irradiation unit that irradiates a target location with the ultraviolet light propagated through the plurality of optical transmission lines.

2. The ultraviolet light irradiation system according to claim 1,
wherein the plurality of optical transmission lines is configured with at least one of:
a multi-core optical fiber;
a first optical cable in which a plurality of single-core optical fibers is bundled; or
a second optical cable in which multi-core optical fibers are bundled, and
at least one of the multi-core optical fiber, the first optical cable, or the second optical cable is connected with a single irradiation unit.

3. The ultraviolet light irradiation system according to claim 2, further comprising a plurality of the irradiation units,
wherein the optical transmission unit is provided for each core included in the multi-core optical fiber, the first optical cable, or the second optical cable and further includes a first optical distribution unit that distributes ultraviolet light propagated by each core included in the multi-core optical fiber, the first optical cable, or the second optical cable into a plurality of parts, and
each of the plurality of irradiation units is connected with the first optical distribution unit and irradiates a target location with each ultraviolet light distributed by the first optical distribution unit.

4. The ultraviolet light irradiation system according to claim 2,
wherein the optical transmission unit further includes a second optical distribution unit that fans out a second optical cable in which multi-core optical fibers are bundled to each multi-core optical fiber included in the second optical cable, and
multi-core optical fibers fanned out by the second optical distribution unit are connected with different irradiation units.

5. The ultraviolet light irradiation system according to claim 1,
wherein at least one of the plurality of optical transmission lines is:
a solid core type optical fiber including a core having a refractive index higher than a refractive index of a clad;
a coupled-core type optical fiber that guides light by optical wave coupling between a plurality of cores;
a hole-assist type optical fiber including a plurality of holes on an outer periphery of a core having a refractive index higher than a refractive index of a clad;
a hole-structure optical fiber that guides light to a region surrounded by holes;
a hollow core type hole-structure optical fiber that guides light to a hollow core surrounded by holes; or
a multi-core optical fiber in which a plurality of at least one of the solid core type optical fibers, coupled-core type optical fibers, hole-assist type optical fibers, hole structure optical fibers, or hollow core type hole structure optical fibers is arranged in a same optical fiber cross section.

6. An ultraviolet light irradiation method characterized by comprising a step of
propagating ultraviolet light to a single irradiation unit using a plurality of optical transmission lines when irradiating a target location with the ultraviolet light outputted from an ultraviolet light source unit from an irradiation unit.
